(19) United States
(12) Patent Application Publication
(10) Pub. No.: US 2022/0175728 A1
(43) Pub. Date: Jun. 9, 2022

(54) PHARMACEUTICAL USE OF CHLOROPHYLLIDE FOR ANTI-VIRAL INFECTION

(71) Applicant: I-SHOU UNIVERSITY, Kaohsiung City (TW)

(72) Inventors: Chih-Hui YANG, Kaohsiung City (TW); Jei-Fu SHAW, Kaohsiung City (TW); Yi-Jung HO, Kaohsiung City (TW)

(21) Appl. No.: 17/537,527

(22) Filed: Nov. 30, 2021

(30) Foreign Application Priority Data
Dec. 7, 2020 (TW) 109143031

(51) Int. Cl. A61K 31/409; A61P 31/14

(52) U.S. Cl. CPC A61K 31/409; A61K 2236/31; A61P 31/14

(57) ABSTRACT

The present invention provides a method for treating or preventing virus infection, the method including the step of: administering a therapeutically effective concentration of chlorophyllide to a subject in need thereof. The present invention further provides a method for treating or preventing virus infection, the method including the step of: administering a product obtained by treating a plant leaf extract with chlorophyllase to a subject in need thereof, wherein the product comprises a therapeutically effective concentration of chlorophyllide.

### CROSS REFERENCE

This non-provisional application claims priority of Taiwan Invention Patent Application No. 109143031, filed on Dec. 7, 2020, the contents thereof are incorporated by reference herein.

### FIELD OF THE INVENTION

The present invention is directed to pharmaceutical use of a chlorophyll derivative for anti-viral infection, and more particularly to pharmaceutical use of chlorophyllide for anti-viral infection.

### BACKGROUND OF THE INVENTION

Plants are the foundation of traditional medicines. A number of plant extracts possess anti-cancer properties, including Annona muricata L., Carica papaya, Colocasia gigantea, Annona squamosa Linn, Murraya koenigii L., Olea europaea L., Pandanus amaryllifolius Roxb., Chenopodium quinoa, Toona sinensis, Myristica fragrans, Thermopsis rhombifolia, and Cannabis sativa. The potential anti-cancer activities of these plants are associated with various bioactive compounds, including chlorophyll, pheophorbide, alkaloid, terpenoid, polysaccharide, lactone, flavonoid, carotenoid, glycoside, and cannabidiol. Beside the possibility of anti-cancer functions, compounds in plant extract demonstrate to exert function of anti-oxidation, anti-inflammation and attenuate side effects induced by chemotherapeutics. Additionally, those bioactive factors, especially chlorophyll and its derivatives, demonstrate potential for the treatment of cancer.

Chlorophyll, the most abundant pigment on earth, is present at high levels in green leafy plants, algae, and cyanobacteria. The catabolic derivatives of chlorophyll are chlorophyllide (chlide), pheophytin, pheophorbide, and phytol. Studies demonstrate that chlorophyll can reduce the growth and proliferation of MCF-7 breast carcinoma cells. Chlorophyll is also reported to promote cell differentiation, and to induce cell cycle arrest and apoptosis in HCT116 colon cancer cells. Chlorophyllide a/b and pheophorbide a/b are reported to reduce hydrogen peroxide-induced strand breaks and oxidative damage, and aflatoxin B1-DNA adduct formation in hepatoma cells. Chlorophyllide is shown to decrease the levels of hepatitis B virus without affecting cell viability and viral gene products in tetracycline-inducible HBV-expressing HepDE19 cells. In human lymphoid leukemia molt 4B cells, pheophorbide a and phytol are able to induce programmed cell death. Phytol can also reduce inflammation by inhibiting neutrophil migration, reducing the levels of interleukin-1β (IL-1β), tumor necrosis factor-α (TNF-α), and oxidative stress. Pheophorbide a, in photodynamic therapy, is found to increase the levels of cytosolic cytochrome c, and is also tested against human pancreatic cancer cells (Panc-1, Capan-1, and HA-hpc2), hepatocellular carcinoma cells (Hep 3B), uterine sarcoma cells, human uterine carcinoma cells, and Jurkat leukemia cells. Pheophorbide is also shown to decrease the levels of procaspase-3 and -9 in Hep3B, Hep G2, and human uterine sarcoma MES-SA cells.

Extensive studies are performed with chlorophyllin (chllin, Cu-chl). Chlorophyllin, a semisynthetic, Cu-coupled, and water-soluble derivative of chlorophyll, is shown to significantly decrease the growth of mutagen-induced cancer cells. In vitro and in vivo studies are suggested that chlorophyllin possesses anti-genotoxic functions against compounds present in cooked meat, including N-nitroso compound and fungal toxin, aflatoxin B1 (AFB1), and dibenzo[d,e,f,p]chrysene (DBC). The regulation of cancer growth by chlorophyllin seems to involve the deactivation of key signal transduction pathways, including the nuclear factor kappa B, Wnt/b-catenin, phosphatidylinositol-3-kinase/Akt, and expressed E-cadherin and alkaline phosphatase pathways.

The amount of chlorophyll degraded globally each year is estimated to exceed 1000 million tons, and this is mostly derived from agriculture and food processing waste. Except for the edible parts of vegetables and fruits, most chlorophyll from low-value agricultural waste can only be degraded naturally. By using low-value agricultural waste as sources to collect chlorophyll, the cost of extraction can be reduced and maximum value of agriculture waste can be reached. Therefore, agricultural waste is potentially useful in the biomedical industry as a high-value nutraceutical and pharmaceutical material.

### SUMMARY OF THE INVENTION

The present invention is made based on the discovery that a product obtained by treating a plant leaf extract with chlorophyllase exhibits anti-viral properties, wherein its active ingredient at least includes chlorophyllide.

Therefore, an embodiment of the present invention provides a method for treating or preventing virus infection, the method including the step of: administering a therapeutically effective concentration of chlorophyllide to a subject in need thereof.

Preferably, the therapeutically effective concentration is 1.5625 μg/mL to 100 μg/mL.

Preferably, the therapeutically effective concentration is 6.25 μg/mL to 100 μg/mL.

Preferably, the therapeutically effective concentration is 25 μg/mL to 100 μg/mL.

Preferably, the virus is flavivirus.

Preferably, the flavivirus is yellow fever virus, Japanese encephalitis virus, dengue virus, West Nile virus, or Zika virus.

Preferably, the virus is alphavirus.

Preferably, the alphavirus is chikungunya virus.

Preferably, the virus is severe acute respiratory syndrome coronavirus 2.

Another embodiment of the present invention provides a method for treating or preventing virus infection, the method including the step of: administering a product obtained by treating a plant leaf extract with chlorophyllase to a subject in need thereof, wherein the product comprises a therapeutically effective concentration of chlorophyllide.

Preferably, the product is produced by the following steps of: providing plant leaves; performing extraction on the plant leaves with a solvent to obtain a crude extract; and treating the crude extract with chlorophyllase to obtain the product.

Preferably, the therapeutically effective concentration is 1.5625 μg/mL to 100 μg/mL.

Preferably, the therapeutically effective concentration is 6.25 μg/mL to 100 μg/mL.

Preferably, the therapeutically effective concentration is 25 μg/mL to 100 μg/mL.

Preferably, the solvent is ethanol or hexane.

Preferably, the virus is flavivirus.

Preferably, the flavivirus is yellow fever virus, Japanese encephalitis virus, dengue virus, West Nile virus, or Zika virus.

Preferably, the virus is alphavirus.

Preferably, the alphavirus is chikungunya virus.

Preferably, the virus is severe acute respiratory syndrome coronavirus 2.

### BRIEF DESCRIPTION OF THE DRAWINGS

FIGS. 1A to 1C show the chlorophyll and chlorophyllide in products obtained by treating ethanol crude extracts from various plant leaves with chlorophyllase by using HPLC;

FIG. 2A shows the effect of various concentrations of chlorophyllide on chikungunya virus infection by using RT-qPCR;

FIG. 2B shows the effect of various concentrations of chlorophyllide on chikungunya virus spread by using the TCID₅₀ assay;

FIG. 2C is a picture showing the effect of various concentrations of chlorophyllide on chikungunya virus micro-neutralization;

FIG. 2D is a graph quantitatively showing the effect on chikungunya virus micro-neutralization as shown in FIG. 2C;

FIG. 3 shows the number of differentially expressed genes in African green monkey kidney Vero cells infected with chikungunya virus relative to reference African green monkey kidney Vero cells, and that in African green monkey kidney Vero cells infected with chikungunya virus in combination with chlorophyllide treatment relative to African green monkey kidney Vero cells infected with chikungunya virus by using next generation sequencing;

FIG. 4A shows the classification of differentially expressed genes in African green monkey kidney Vero cells infected with chikungunya virus relative to reference African green monkey kidney Vero cells by using the Gene Ontology analysis;

FIG. 4B shows the classification of differentially expressed genes in African green monkey kidney Vero cells infected with chikungunya virus in combination with chlorophyllide treatment relative to African green monkey kidney Vero cells infected with chikungunya virus by using the Gene Ontology analysis;

FIG. 5A shows the classification of differentially expressed genes in African green monkey kidney Vero cells infected with chikungunya virus relative to reference African green monkey kidney Vero cells by using the KEGG pathway enrichment analysis;

FIG. 5B shows the classification of differentially expressed genes in African green monkey kidney Vero cells infected with chikungunya virus in combination with chlorophyllide treatment relative to African green monkey kidney Vero cells infected with chikungunya virus by using the KEGG pathway enrichment analysis;

FIG. 6A shows the effect of various concentrations of chlorophyllide on Zika virus infection by using RT-qPCR;

FIG. 6B shows the effect of various concentrations of chlorophyllide on Zika virus spread by using the TCID₅₀ assay;

FIG. 6C is a picture showing the effect of various concentrations of chlorophyllide on Zika virus micro-neutralization;

FIG. 6D is a graph quantitatively showing the effect on Zika virus micro-neutralization as shown in FIG. 6C;

FIG. 7A shows the effect of various concentrations of chlorophyllide on dengue-2 virus infection by using RT-qPCR;

FIG. 7B shows the effect of various concentrations of chlorophyllide on dengue-2 virus spread by using the TCID₅₀ assay;

FIGS. 8A to 8B show the number of differentially expressed genes in African green monkey kidney Vero cells infected with dengue virus relative to reference African green monkey kidney Vero cells, and that in African green monkey kidney Vero cells infected with dengue virus in combination with chlorophyllide treatment relative to African green monkey kidney Vero cells infected with dengue virus by using next generation sequencing;

FIG. 8C shows the interactions of differentially expressed genes as shown in FIGS. 8A to 8B by using the String analysis;

FIG. 9A shows the classification of differentially expressed genes in African green monkey kidney Vero cells infected with dengue virus relative to reference African green monkey kidney Vero cells by using the Gene Ontology analysis;

FIG. 9B shows the classification of differentially expressed genes in African green monkey kidney Vero cells infected with dengue virus in combination with chlorophyllide treatment relative to African green monkey kidney Vero cells infected with dengue virus by using the Gene Ontology analysis;

FIG. 10A shows the classification of differentially expressed genes in African green monkey kidney Vero cells infected with dengue virus relative to reference African green monkey kidney Vero cells by using the KEGG pathway enrichment analysis;

FIG. 10B shows the classification of differentially expressed genes in African green monkey kidney Vero cells infected with dengue virus in combination with chlorophyllide treatment relative to African green monkey kidney Vero cells infected with dengue virus by using the KEGG pathway enrichment analysis;

FIG. 11A shows the classification of differentially expressed genes in African green monkey kidney Vero cells infected with dengue virus relative to reference African green monkey kidney Vero cells by using the KEGG pathway enrichment analysis;

FIG. 11B shows the classification of differentially expressed genes in African green monkey kidney Vero cells infected with dengue virus in combination with chlorophyllide treatment relative to African green monkey kidney Vero cells infected with dengue virus by using the KEGG pathway enrichment analysis;

FIG. 12A shows the regulation of protein processing in endoplasmic reticulum by differentially expressed genes in African green monkey kidney Vero cells infected with dengue virus relative to reference African green monkey kidney Vero cells, wherein the red labels denote positive regulated genes, and the green labels denote negative regulated genes; and

FIG. 12B shows the regulation of protein processing in endoplasmic reticulum by differentially expressed genes in African green monkey kidney Vero cells infected with dengue virus in combination with chlorophyllide treatment relative to African green monkey kidney Vero cells infected with dengue virus, wherein the red labels denote positive regulated genes, and the green labels denote negative regulated genes.

### DETAILED DESCRIPTION OF THE INVENTION

The detailed description and preferred embodiments of the invention will be set forth in the following content, and provided for people skilled in the art to understand the characteristics of the invention.

The compound chlorophyllide used in the content is represented by a general formula (I)

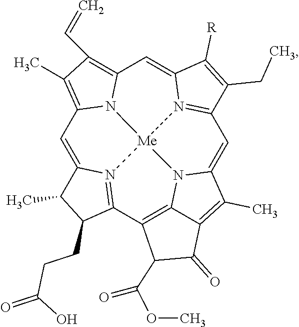

in which Me is a Mg atom, and R is a CH₃ group or a CHO group. Generally, while R is a CH₃ group, the compound is called “chlorophyllide a”; while R is a CHO group, the compound is called “chlorophyllide b.”

### Example 1: Preparation of Crude Extracts and Chlorophyll Extraction

The leaves of guava, sweet potato, banana, Chinese toona, logan, wax apple, mango, caimito, and cocoa were used to extract chlorophyll. 10 g (wet weight) of leaves were washed, dried, and ground into powder with a pestle and mortar. Leaf mixtures were then frozen in liquid nitrogen and stored at −80° C. in a deep freezer. Chlorophyll was extracted by immersing leaves in ethanol solvent (or hexane solvent) for 48 h. Ethanol crude extracts (or hexane crude extracts) from leaves were centrifuged at 1500 g for 5 min and keep at −20° C. for further experiments. To measure the concentrations of chlorophyll a/b, the crude extracts were passed through a 0.22-μm filter and the absorbance was measured at 649 and 665 nm, which were the major absorption peaks of chlorophyll a and b, respectively. The estimated concentrations of chlorophyll a and b in crude extracts were calculated according to the following equation:

chlorophyll a concentration(μg/mL)=13.7×A665−5.76×A649;

chlorophyll b concentration(μg/mL)=25.8×A665−7.6×A649.

The chlorophyll a/b concentrations in crude extracts calculated with the empirical equation were multiplied by the volume of the solvent that resulted in the relative chlorophyll mass values in the given samples. When the dry and wet weights of the plant species are known, the content of chlorophyll a/b and the mass of crude extracts relative to the mass of the dry plant can be calculated and expressed as mg/gDW.

### Example 2: Preparation of Chlorophyllase-Treated Plant Leaf Extracts

Chlamydomonas reinhardtii chlorophyllase was produced as described previously (Molecules. 2015 Feb. 24; 20(3):3744-57; Biotechnol Appl Biochem. 2016 May; 63(3):371-7). Recombinant Chlamydomonas reinhardtii chlorophyllase was expressed, purified, and then lyophilized. The reaction mixture contained 0.5 mg of recombinant chlorophyllase, 650 μL of the reaction buffer (100 mM sodium phosphate, pH 7.4, and 0.24% Triton X-100), and 0.1 ml of crude extracts from leaves (100 mM chlorophyll). The reaction mixture was incubated at 37° C. for 30 min in a shaking water bath. The enzymatic reaction was stopped by adding 4 mL of ethanol, 6 mL of hexane, and 1 mL of 10 mM KOH, respectively. The reaction mixture was vortexed vigorously and centrifuged at 4000 rpm for 10 min to separate the two phases. The upper layer contained the untreated chlorophyll a/b; the bottom layer was chlorophyllase-treated crude extracts comprising chlorophyllide a/b. The chlorophyllase-treated crude extracts containing chlorophyllide a/b mixtures were then concentrated and the solvent was removed by evaporation under reduced pressure at 40° C. on a rotary evaporator. The concentrated crude extracts were processed by lyophilization, weighed, and stored at −80° C. for further experiments.

Chlorophyll was extracted from leaves of 9 plant species, including guava, sweet potato, banana, Chinese toona, logan, wax apple, mango, caimito, and cocoa. Ethanol crude extracts were treated with chlorophyllase to generate chlorophyllide, and then lyophilized in order to measure the weight. The results are listed in Table 1. Significantly, the most chlorophyll a level was observed in Chinese toona (9.8 mg/gDW), followed by mango (8.407 mg/gDW). The lowest chlorophyll a levels were present in banana (2.921 mg/gDW) and sweet potato (3.481 mg/gDW). For chlorophyll b, Chinese toona possessed the highest content (5.419 mg/gDW), followed by cocoa (4.485 mg/gDW) and mango (2.599 mg/gDW). The lowest levels of chlorophyll b were found in sweet potato (0.996 mg/gDW), banana (1.031 mg/gDW), and caimito (1.493 mg/gDW). Of the species analyzed, leaves of cocoa and caimito contained the highest level of ethanol crude extracts, at 412.65 and 397.62 mg/gDW, respectively. The lowest weight of ethanol crude extracts was obtained from sweet potato (43.175 mg/gDW), banana (47.76 mg/gDW), and wax apple (94.29 mg/gDW).

TABLE 1
The concentration of chlorophyll a/b extracted from leaves of plants

| | | | | Chlorophyllase- |
|---|---|---|---|---|
| | | | | treated crude |
| | | | | extracts containing |
| | | Chlorophyll a | Chlorophyll b | chlorophyllide a/b |
| Plant species | | (mg/gDW) | (mg/gDW) | (mg/gDW) |
| Sweet potato | Ipomoea batatas | 3.481 | 0.996 | 43.17 |
| Wax apple | Syzygium | 5.423 | 1.955 | 94.29 |
| | samarangense | | | |
| Guava | Psidium guajava | 5.219 | 1.493 | 124.39 |
| Banana | Musa paradisiaca | 2.921 | 1.031 | 47.76 |
| Chinese toona | Toona sinensis | 9.800 | 5.419 | 148.19 |
| Logan | Dimocarpus longan | 7.044 | 1.903 | 183.15 |
| Mango | Mangifera indica | 8.407 | 2.599 | 291.77 |
| Caimito | Pouteria Caimito | 5.218 | 1.493 | 397.62 |
| Cocoa | Theobroma cacao | 6.718 | 4.485 | 412.65 |

### Example 3: High-Performance Liquid Chromatography (HPLC) Analysis of Chlorophyll Catabolites

To analyze chlorophyll and chlorophyllide, the mixtures containing chlorophyllase-treated crude extracts were analyzed by using HPLC as described previously. Chlorophyllide was detected at a wavelength of 667 nm and identified by absorption spectra, peak ratios, and co-migration with authentic standards.

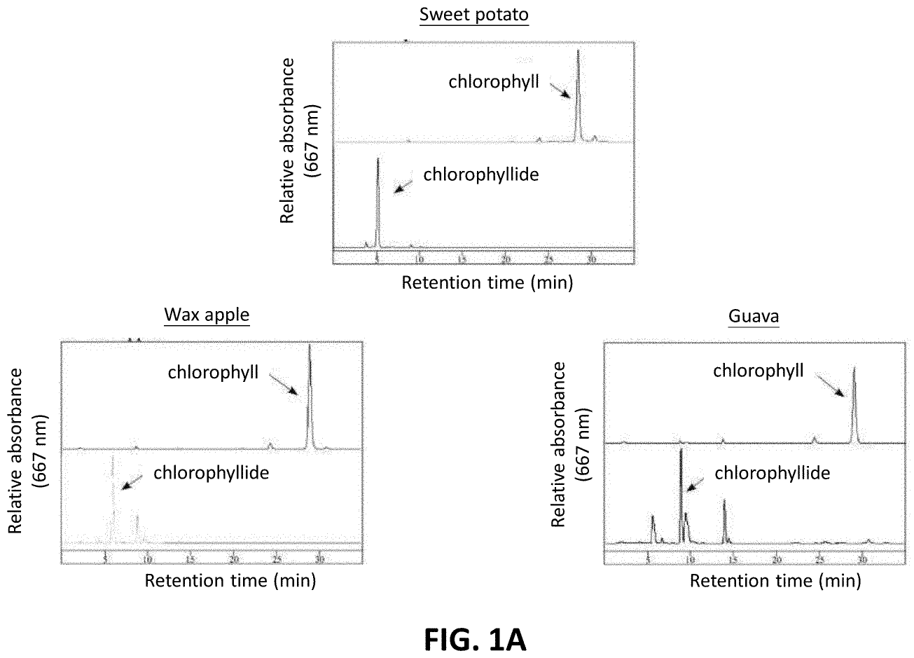

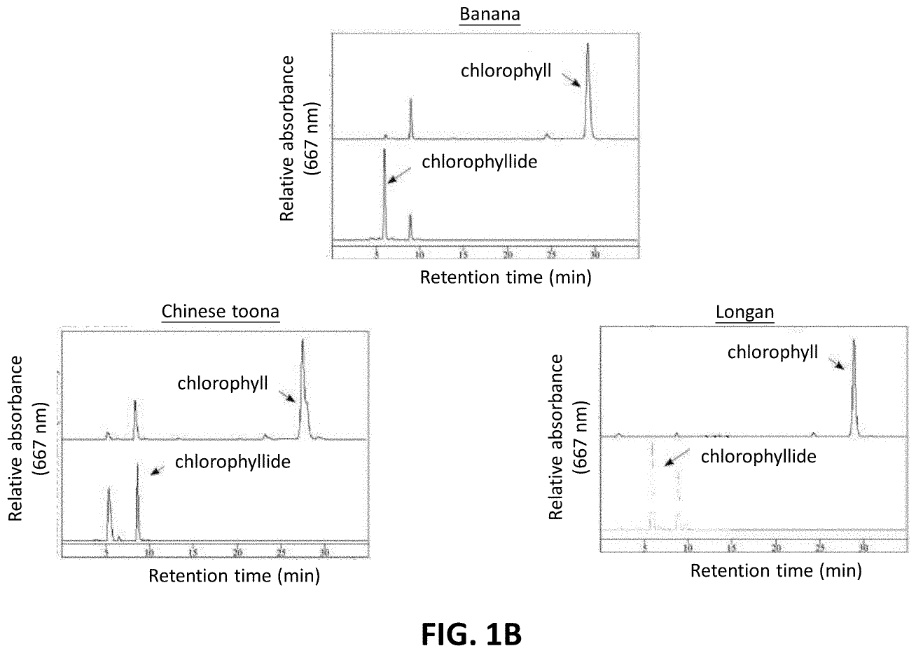

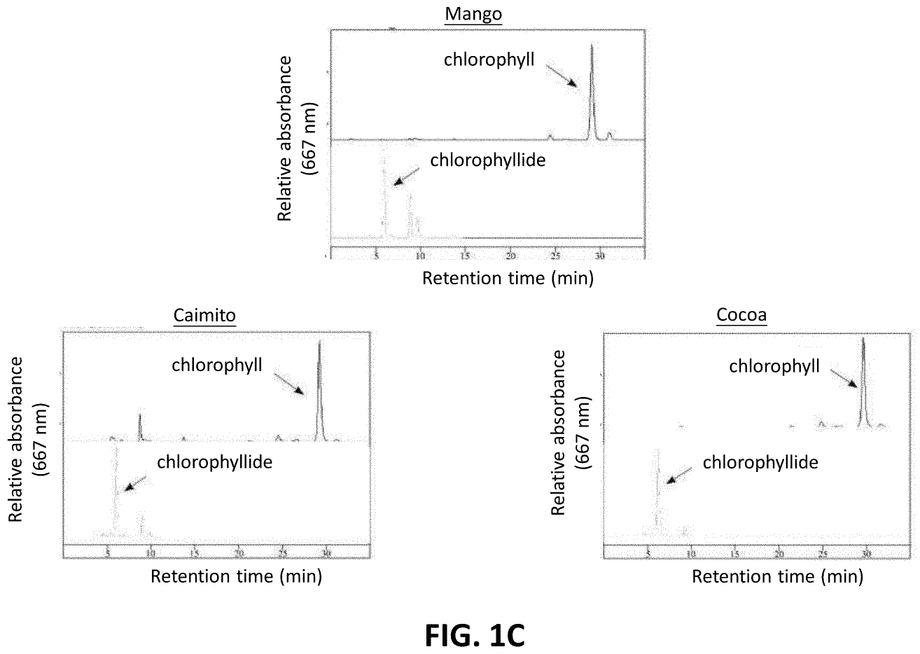

The HPLC separation system was applied to determine the amount of chlorophyll a/b and chlorophyllide a/b in crude extracts. Since the provision of commercial standards was limited, it was not possible to identify all peaks in all crude extracts by HPLC. Herein, the standards used in this study, including chlorophyll a, chlorophyll b, chlorophyllide a, and chlorophyllide b were selected based on our previous studies. HPLC results were obtained using mobile phases consisting of ethyl acetate/methanol/H₂O₂=44:50:6. Samples were quantified using photodiode array detection in the region 200-400 nm based on the retention times and UV spectra compared with the standards. FIGS. 1A to 1C show the HPLC profiles of guava, sweet potato, banana, Chinese toona, longan, wax apple, mango, caimito, and cocoa, respectively. The solvent system identified chlorophyll from 9 plant species within 30 min with a flow rate at 1 mL/min and detection at 667 nm. Chlorophyllide in crude extracts was detected within 10 min at 667 nm. According to the retention time, standards, and UV spectra, the peaks in FIGS. 1A to 1C were identified as chlorophyll and chlorophyllide.

### Example 4: Antiviral Assay Against Chikungunya

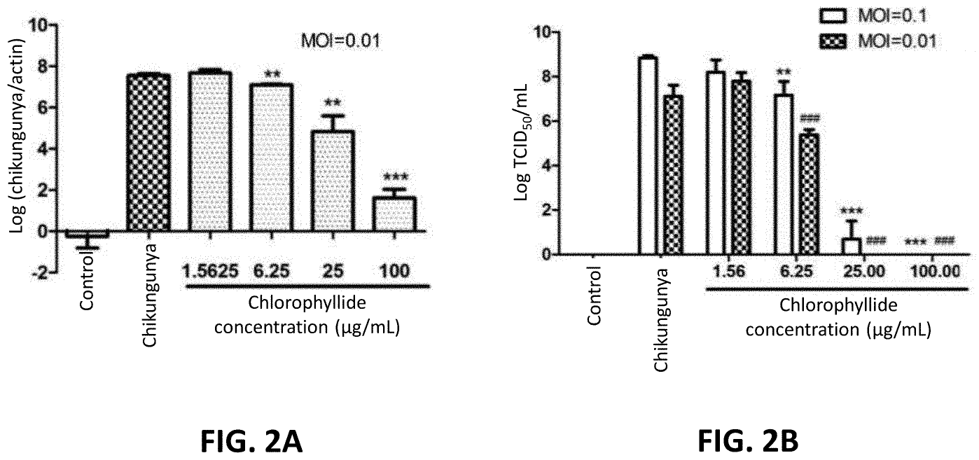

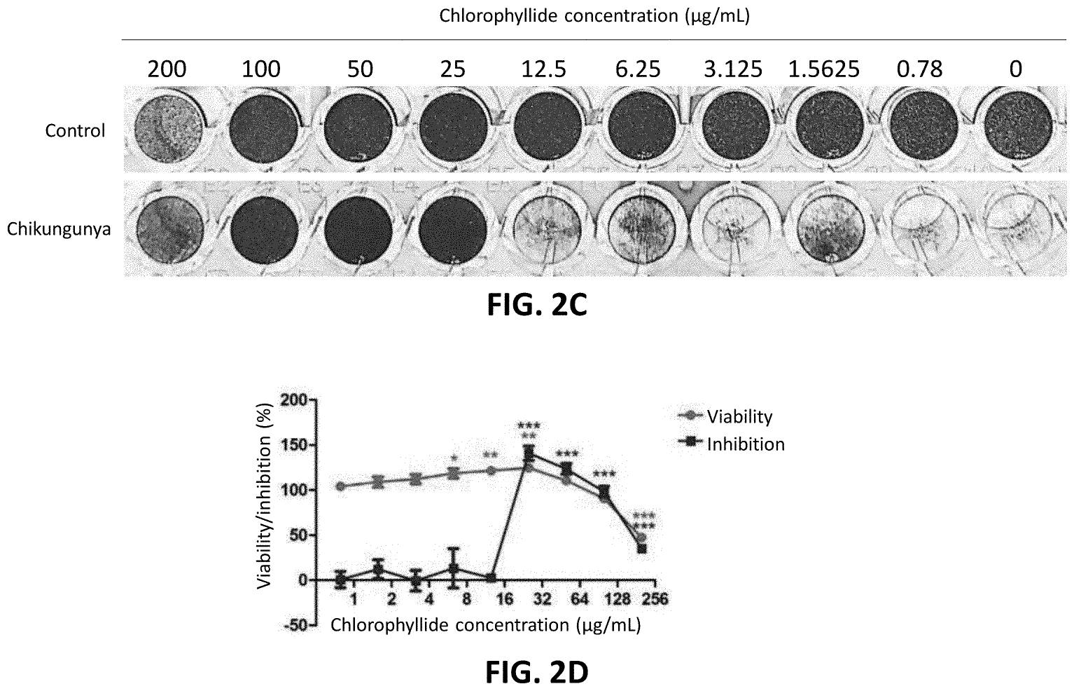

African green monkey kidney Vero cells were infected with chikungunya virus at a multiplicity of infection (MOI) of 0.1 or 0.01 in the presence of chlorophyllide at indicated dosages, and then incubated for 24 hr. Total RNA in the infected Vero cells was isolated with NucleoZOL (Macherey-Nagel) and quantified by RT-qPCR using Mic qPCR (Bio Molecular Systems). The QuantiTech SYBR Green RT-qPCR kit was used to prepare mixture samples, followed by a program of 50° C. for 30 min, 95° C. for 15 min, and then 45 cycles at 95° C. for 15 sec, 57° C. for 25 sec, and 72° C. for 10 sec. Meanwhile, after incubation with chlorophyllide for 24 hr, the culture supernatant was collected to determine virus spread by using the TCID₅₀ assay. FIGS. 2A to 2B show chlorophyllide obviously decreases chikungunya RNA levels and inhibits chikungunya spread at a concentration of 6.25 μg/mL to 100 μg/mL.

African green monkey kidney Vero cells were infected with chikungunya virus at an MOI of 0.001 in the presence of chlorophyllide at indicated dosages, and then incubated for 3 days, followed by the micro-neutralization test. FIG. 2C shows the control group are used to analyze the cytotoxicity of chlorophyllide and the chikungunya-infected group are used to analyze the antiviral activity against chikungunya of chlorophyllide. Quantitative result from FIG. 2C is shown in FIG. 2D. According to the quantitative result, the cytotoxicity dose of chlorophyllide is greater than 100 μg/mL; the antiviral dose against chikungunya of chlorophyllide is 25 μg/mL to 100 μg/mL, and the EC₅₀ for antiviral activity against chikungunya of chlorophyllide is 18.75 μg/mL.

The gene expression profiles of the African green monkey kidney Vero cells infected with chikungunya and those infected with chikungunya in combination with chlorophyllide treatment were analyzed by using next generation sequencing (NGS). Table 2 shows both test samples are good samples according to quality evaluation in NGS data.

TABLE 2
Quality Evaluation in NGS data

| | | Vero cell infected |
|---|---|---|
| | | with chikungunya in |
| | Vero cells infected | combination with |
| Sample type | with chikungunya | chlorophyllide treatment |
| Total reads | 40600830 | 54478764 |
| Total mapped (%) | 38423761 (94.64%) | 51944999 (95.35%) |
| Multiple mapped (%) | 1071024 (2.64%) | 1516391 (2.78%) |
| Uniquely mapped (%) | 37352737 (92.00%) | 50428608 (92.57%) |
| Clean reads | 40600830 | 54478764 |
| Raw bases (G) | 6.9 | 9.18 |
| Q20 (%) | 97.6 | 97.7 |
| Q30 (%) | 92.2 | 92.4 |
| GC content (%) | 44.7 | 44.7 |

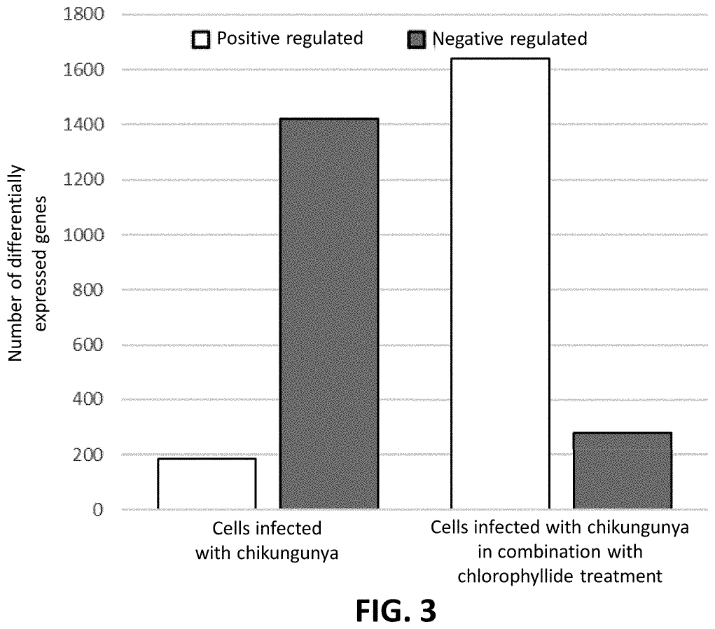

FIG. 3 shows 1600 differentially expressed genes including 200 positive regulated genes and 1400 negative regulated genes are found in African green monkey kidney Vero cells infected with chikungunya relative to reference African green monkey kidney Vero cells, and 1930 differentially expressed genes including 1650 positive regulated genes and 280 negative regulated genes are found in African green monkey kidney Vero cells infected with chikungunya in combination with chlorophyllide treatment relative to African green monkey kidney Vero cells infected with chikungunya.

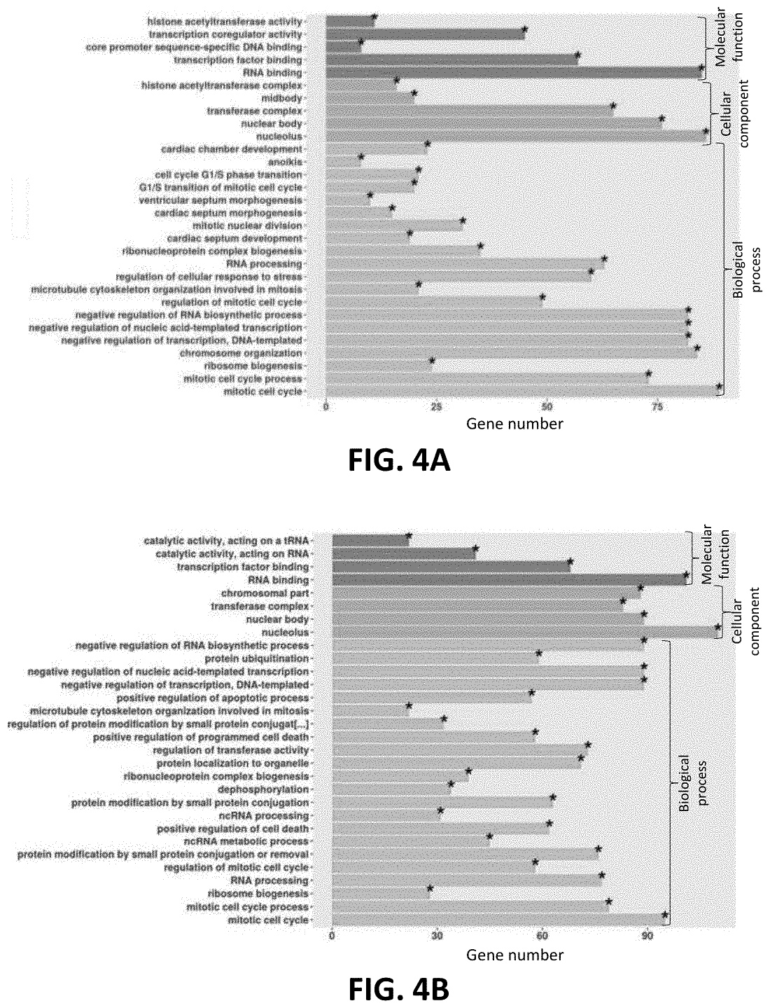

The Gene Ontology analysis was performed to classify the foregoing 2 differentially expressed gene groups. Based on sequence homology, FIG. 4A shows that the differentially expressed gene group identified from African green monkey kidney Vero cells infected with chikungunya relative to reference African green monkey kidney Vero cells are classified into the subdomain “mitotic cell cycle.” FIG. 4B shows that the differentially expressed gene group identified from African green monkey kidney Vero cells infected with chikungunya in combination with chlorophyllide treatment relative to African green monkey kidney Vero cells infected with chikungunya are classified into the subdomain “nucleolus.”

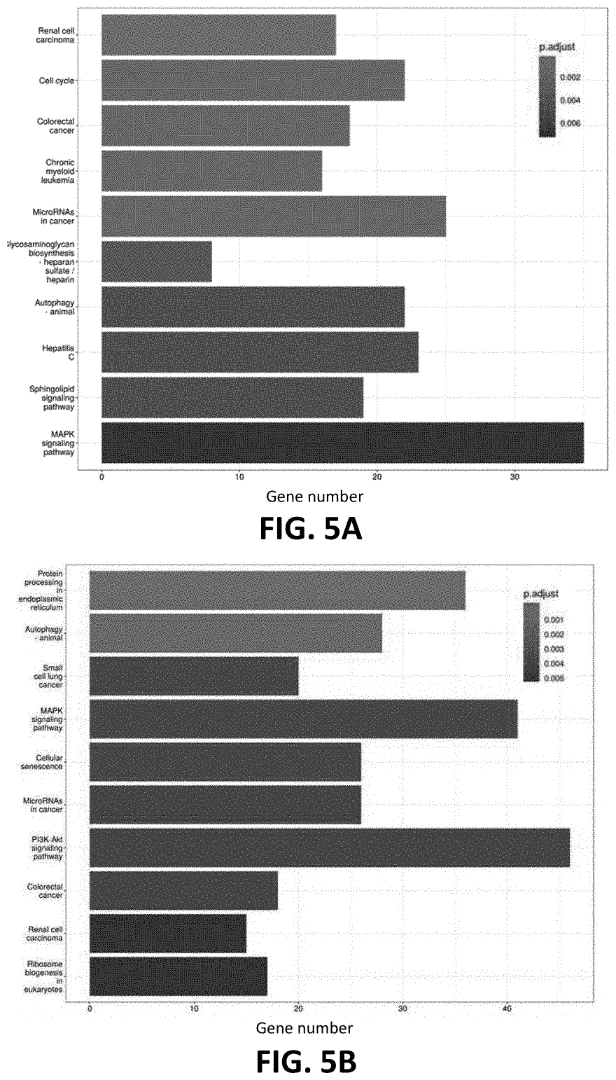

The KEGG pathway enrichment analysis was also performed to classify the foregoing 2 differentially expressed gene group. In the KEGG pathway enrichment analysis, all of the foregoing differentially expressed genes were classified according to different KEGG pathways including “metabolism”, “genetic information processing”, “environmental information processing”, “cellular processes”, “organismal systems”, and “human diseases.” FIG. 5A shows that the differentially expressed genes identified from African green monkey kidney Vero cells infected with chikungunya relative to reference African green monkey kidney Vero cells are most relevant to the MAPK signaling pathway; FIG. 5B shows that the differentially expressed genes identified from African green monkey kidney Vero cells infected with chikungunya in combination with chlorophyllide treatment relative to African green monkey kidney Vero cells infected with chikungunya are most relevant to the PI3-Akt signaling pathway.

### Example 5: Antiviral Assay Against Zika

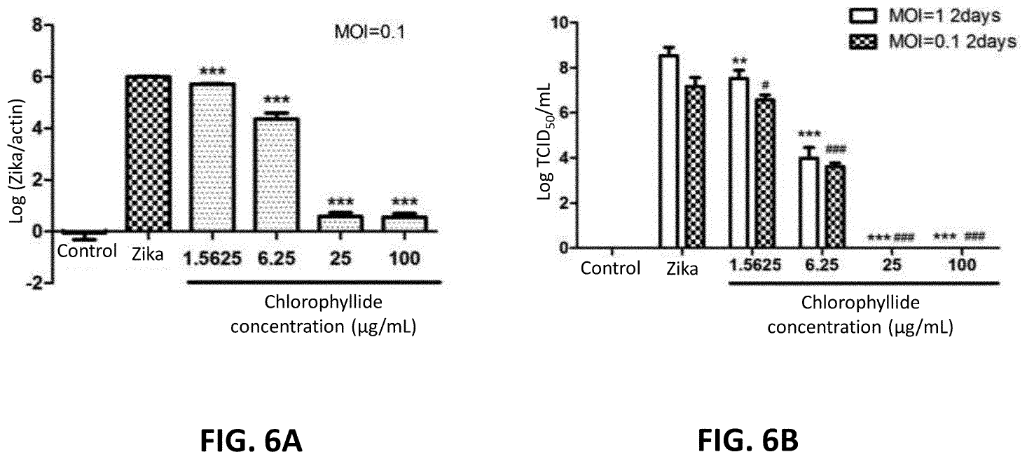

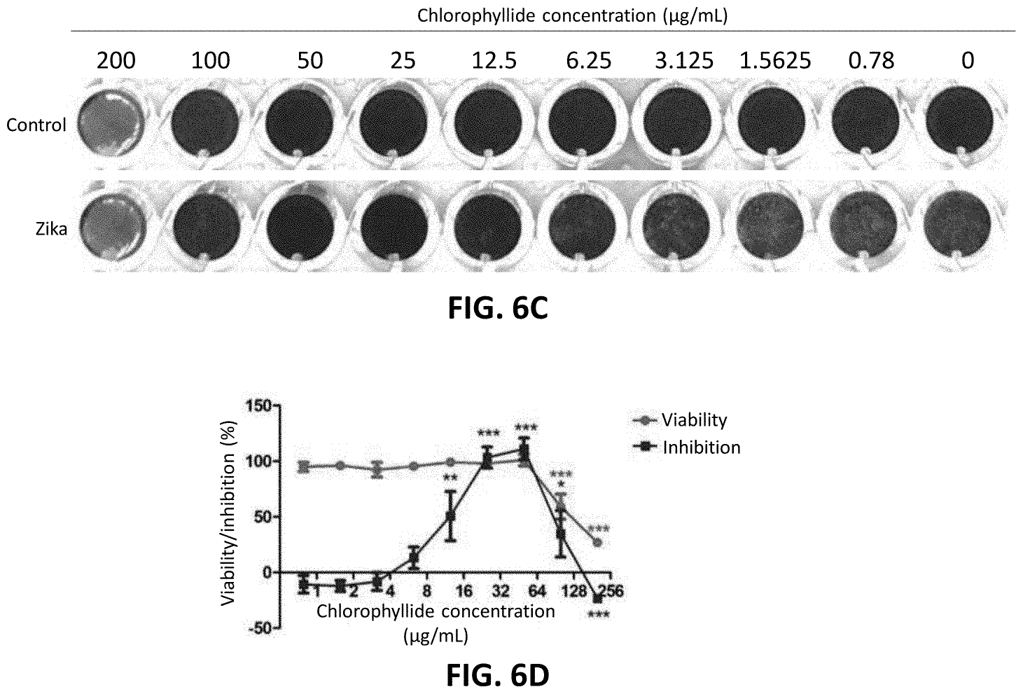

African green monkey kidney Vero cells were infected with Zika virus at an MOI of 1 or 0.1 in the presence of chlorophyllide at indicated dosages, and then incubated for 2 days. Total RNA in the infected Vero cells was isolated with NucleoZOL (Macherey-Nagel) and quantified by RT-qPCR using Mic qPCR (Bio Molecular Systems). The QuantiTech SYBR Green RT-qPCR kit was used to prepare mixture samples, followed by a program of 50° C. for 30 min, 95° C. for 15 min, and then 45 cycles at 95° C. for 5 sec, and 60° C. for 34 sec. Meanwhile, after incubation with chlorophyllide for 2 days, the culture supernatant was collected to determine virus spread by using the TCID₅₀ assay. FIGS. 6A to 6B show chlorophyllide obviously decreases Zika RNA levels and inhibits Zika spread at a concentration of 1.5625 μg/mL to 100 μg/mL.

African green monkey kidney Vero cells were infected with Zika virus at an MOI of 0.1 in the presence of chlorophyllide at indicated dosages, and then incubated for 4 to 5 days, followed by the micro-neutralization test. FIG. 6C shows the control group are used to analyze the cytotoxicity of chlorophyllide and the Zika-infected groups are used to analyze the antiviral activity against Zika of chlorophyllide. Quantitative result from FIG. 6C is shown in FIG. 6D. According to the quantitative result, the cytotoxicity dose of chlorophyllide is greater than 100 μg/mL; the antiviral dose against Zika of chlorophyllide is 6.25 μg/mL to 100 μg/mL, and the EC₅₀ for antiviral activity against Zika of chlorophyllide is 12.5 μg/mL.

### Example 6: Antiviral Assay Against Dengue

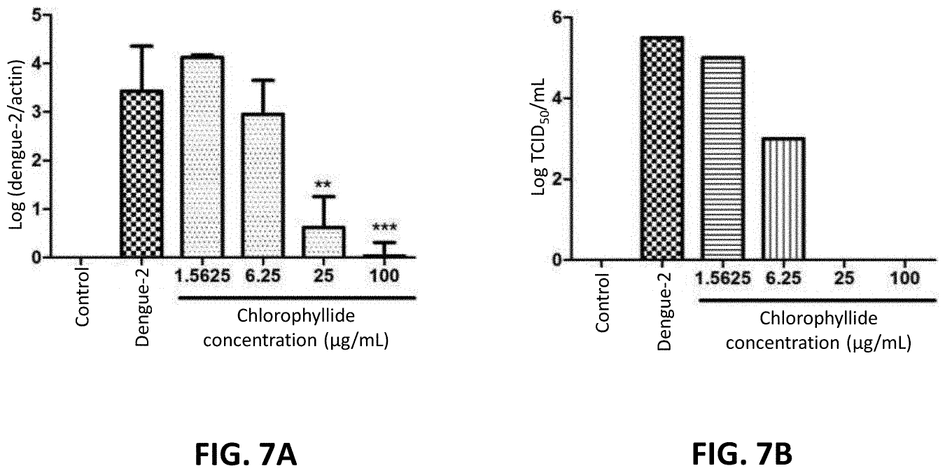

African green monkey kidney Vero cells were infected with dengue-2 virus at an MOI of 0.1 in the presence of chlorophyllide at indicated dosages, and then incubated for 3 days. Total RNA in the infected Vero cells was isolated with NucleoZOL (Macherey-Nagel) and quantified by RT-qPCR using Mic qPCR (Bio Molecular Systems). The QuantiTech SYBR Green RT-qPCR kit was used to prepare mixture samples, followed by a program of 50° C. for 30 min, 95° C. for 15 min, and then 45 cycles at 95° C. for 15 sec, 55° C. for 30 sec, and 72° C. for 30 sec. Meanwhile, after incubation with chlorophyllide for 3 days, the culture supernatant was collected to determine virus spread by using the TCID₅₀ assay. FIGS. 7A to 7B show chlorophyllide obviously decreases dengue RNA levels and inhibits dengue spread at a concentration of 25 μg/mL to 100 μg/mL.

The gene expression profiles of the African green monkey kidney Vero cells infected with dengue and those infected with dengue in combination with chlorophyllide treatment were analyzed by using next generation sequencing (NGS). Table 3 shows both test samples are good samples according to quality evaluation in NGS data.

TABLE 3
Quality Evaluation in NGS data

| | | Vero cell infected |
|---|---|---|
| | | with dengue in |
| | Vero cells infected | combination with |
| Sample type | with dengue | chlorophyllide treatment |
| Total reads | 41063810 | 43064422 |
| Total mapped (%) | 3755812 (9.15%) | 41092066 (95.42%) |
| Multiple mapped (%) | 146452 (0.36%) | 221179 (2.84%) |
| Uniquely mapped (%) | 3609360 (8.79%) | 39870887 (92.58%) |
| Clean reads | 41063810 | 43064422 |
| Raw bases (G) | 7.25 | 7.28 |
| Q20 (%) | 97.7 | 97.7 |
| Q30 (%) | 92.7 | 92.3 |
| GC content (%) | 49.1 | 45.1 |

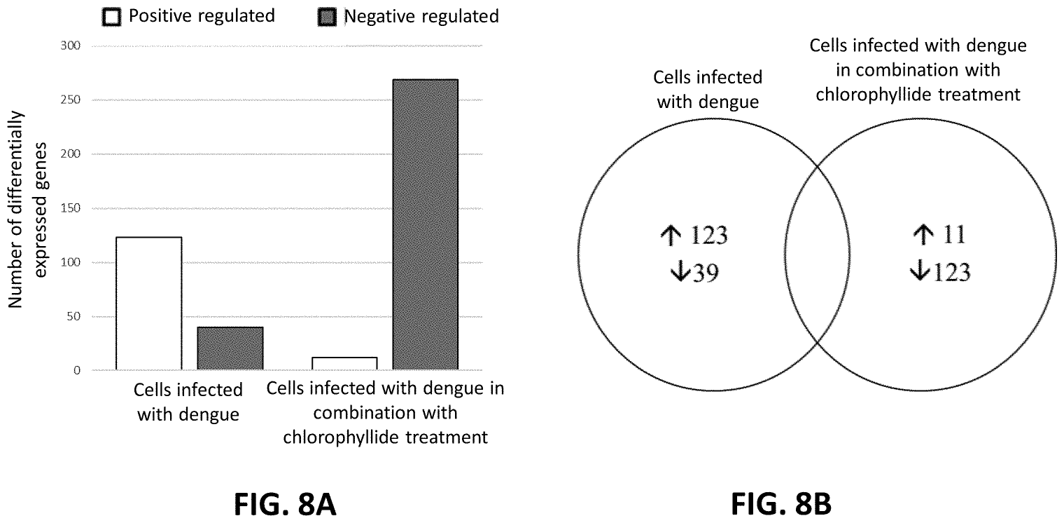

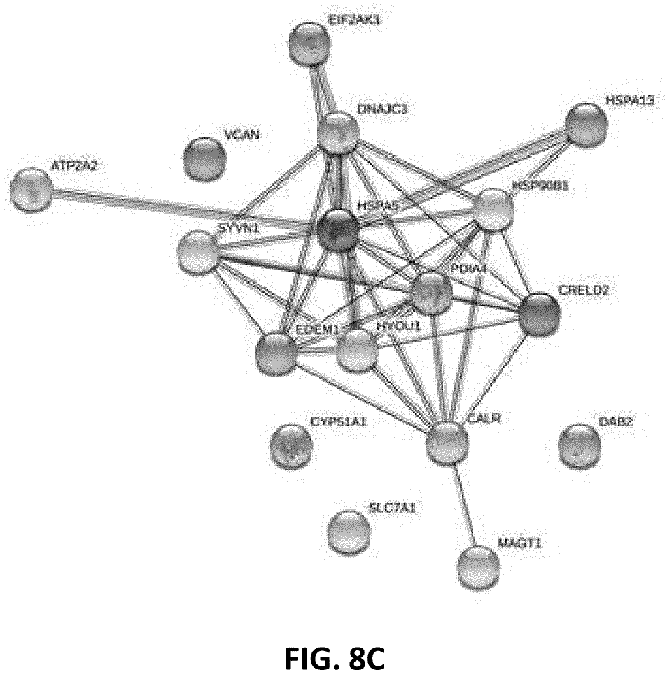

FIG. 8A shows 162 differentially expressed genes including 123 positive regulated genes and 39 negative regulated genes are found in African green monkey kidney Vero cells infected with dengue relative to reference African green monkey kidney Vero cells, and 134 differentially expressed genes including 11 positive regulated genes and 123 negative regulated genes are found in African green monkey kidney Vero cells infected with dengue in combination with chlorophyllide treatment relative to African green monkey kidney Vero cells infected with dengue. Some differentially expressed genes identified from African green monkey kidney Vero cells infected with dengue relative to reference African green monkey kidney Vero cells are listed in Table 4, and some differentially expressed genes identified from African green monkey kidney Vero cells infected with dengue in combination with chlorophyllide treatment relative to African green monkey kidney Vero cells infected with dengue are listed in Table 5. As compared the differentially expressed genes identified from African green monkey kidney Vero cells infected with dengue relative to reference African green monkey kidney Vero cells, and the 134 differentially expressed genes identified from African green monkey kidney Vero cells infected with dengue in combination with chlorophyllide treatment relative to African green monkey kidney Vero cells infected with dengue, FIG. 8A shows that 7 genes are both positively regulated in African green monkey kidney Vero cells infected with dengue relative to reference African green monkey kidney Vero cells and in African green monkey kidney Vero cells infected with dengue in combination with chlorophyllide treatment relative to African green monkey kidney Vero cells infected with dengue, and 1 gene is both negatively regulated in African green monkey kidney Vero cells infected with dengue relative to reference African green monkey kidney Vero cells and in African green monkey kidney Vero cells infected with dengue in combination with chlorophyllide treatment relative to African green monkey kidney Vero cells infected with dengue. FIG. 8C shows the String analysis result made according to the two differentially expressed gene groups.

TABLE 4
Differentially expressed genes identified from Vero cells
infected with dengue relative to reference Vero cells

| | | Log₂ |
|---|---|---|
| Gene | | fold |
| name | Gene description | change |
| DNAJC3 | DnaJ heat shock protein family (Hsp40) member | 2.756 |
| | C3 | |
| IL6 | interleukin 6 | 2.690 |
| TNFAIP3 | TNF alpha induced protein 3 | 2.092 |
| GADD45A | growth arrest and DNA damage inducible alpha | 1.794 |
| CDKN1A | cyclin dependent kinase inhibitor 1A | 1.231 |
| EIF2AK3 | eukaryotic translation initiation factor 2 alpha | 1.156 |
| | kinase 3 | |
| TRIM5 | tripartite motif containing 5 | 1.109 |
| CALR | calreticulin | 1.051 |
| MYC | MYC proto-oncogene | 1.051 |
| RCAN1 | regulator of calcineurin 1 | 1.033 |
| STAT1 | signal transducer and activator of transcription 1 | −1.018 |
| PDGFB | platelet derived growth factor subunit B | −1.172 |
| ITGA4 | integrin subunit alpha 4 | −1.228 |
| MMP1 | matrix metallopeptidase 1 | 2.195 |
| HSP90B1 | heat shock protein 90 beta family member 1 | 1.761 |
| MYLK | myosin light chain kinase | −1.282 |
| VAV3 | vav guanine nucleotide exchange factor 3 | −1.949 |
| DDIT3 | DNA damage inducible transcript 3 | 4.011 |
| GDF15 | growth differentiation factor 15 | 2.716 |
| SESN2 | sestrin 2 | 1.882 |
| DDIT4 | DNA damage inducible transcript 4 | 1.559 |
| SLC7A5 | solute carrier family 7 member 5 | 1.450 |
| ATP2B1 | ATPase plasma membrane Ca²⁺ transporting 1 | 1.376 |
| ATP2A2 | ATPase sarcoplasmic/endoplasmic reticulum Ca²⁺ | 1.370 |
| | transporting 2 | |
| INHBA | inhibin subunit beta A | 1.091 |
| CTSD | cathepsin D | −1.254 |
| VCAN | versican | −1.724 |
| BMP3 | bone morphogenetic protein 3 | −2.663 |
| WIPI1 | WD repeat domain | 1.770 |
| DAB2 | DAB adaptor protein 2 | −1.553 |

TABLE 5
Differentially expressed genes identified from Vero cells
infected with dengue in combination with chlorophyllide
treatment relative to Vero cells infected with dengue

| | | Log₂ |
|---|---|---|
| Gene | | fold |
| name | Gene description | change |
| PDIA3 | protein disulfide isomerase family A member 3 | −1.563 |
| CALR | calreticulin | −1.590 |
| NRP1 | neuropilin 1 | −1.611 |
| COL4A4 | collagen type IV alpha 4 chain | −1.660 |
| EGFR | epidermal growth factor receptor | −1.680 |
| EIF2AK3 | eukaryotic translation initiation factor 2 alpha | −1.719 |
| | kinase 3 | |
| ITGA1 | integrin subunit alpha 1 | −1.762 |
| IL6ST | interleukin 6 signal transducer | −1.880 |
| DNAJC3 | DnaJ heat shock protein family (Hsp40) member | −2.131 |
| | C3 | |
| NOTCH2 | notch receptor 2 | −2.195 |
| HSPG2 | heparan sulfate proteoglycan 2 | −2.242 |
| RELN | reelin | −2.398 |
| TFRC | transferrin receptor | −1.925 |
| HSP90B1 | heat shock protein 90 beta family member 1 | −2.359 |
| PLIN2 | perilipin 2 | 1.440 |
| AGRN | agrin | −1.539 |
| BMPR2 | bone morphogenetic protein receptor type 2 | −1.641 |
| EPHA2 | EPH receptor A2 | −1.661 |
| SCD | stearoyl-CoA desaturase | −1.735 |
| DAG1 | dystroglycan 1 | −1.806 |
| NEO1 | neogenin 1 | −1.905 |
| HMGCR | 3-hydroxy-3-methylglutaryl-CoA reductase | −2.042 |
| ABCC1 | ATP binding cassette subfamily C member 1 | −2.058 |
| ATP2A2 | ATPase sarcoplasmic/endoplasmic reticulum Ca2+ | −2.280 |
| | transporting 2 | |
| VCAN | versican | −3.721 |
| FN1 | fibronectin 1 | −3.808 |
| FTL | ferritin light chain | 1.194 |
| BMPR2 | bone morphogenetic protein receptor type 2 | −1.641 |
| ACSL3 | acyl-CoA synthetase long chain family member 3 | −1.699 |
| SLC7A1 | solute carrier family 7 member 1 | −2.059 |

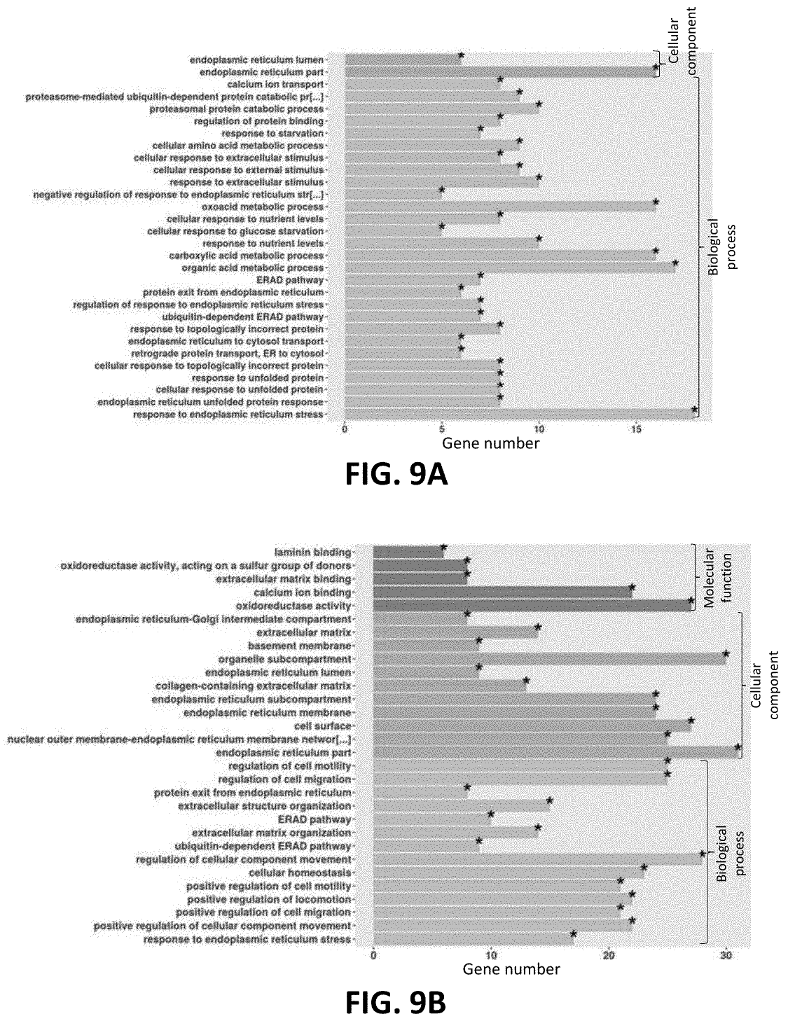

The Gene Ontology analysis was performed to classify the foregoing 2 differentially expressed gene groups. FIG. 9A to 9B show that the foregoing 2 differentially expressed gene groups are classified into the subdomain “response to endoplasmic reticulum stress.”

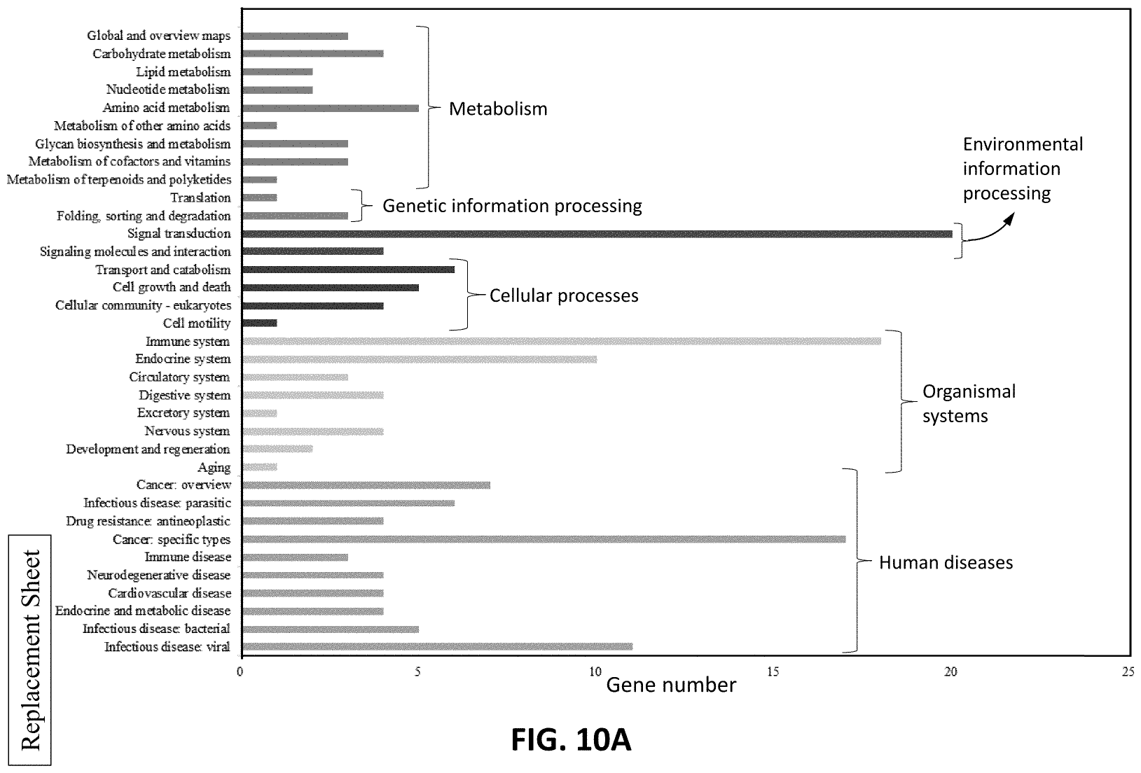

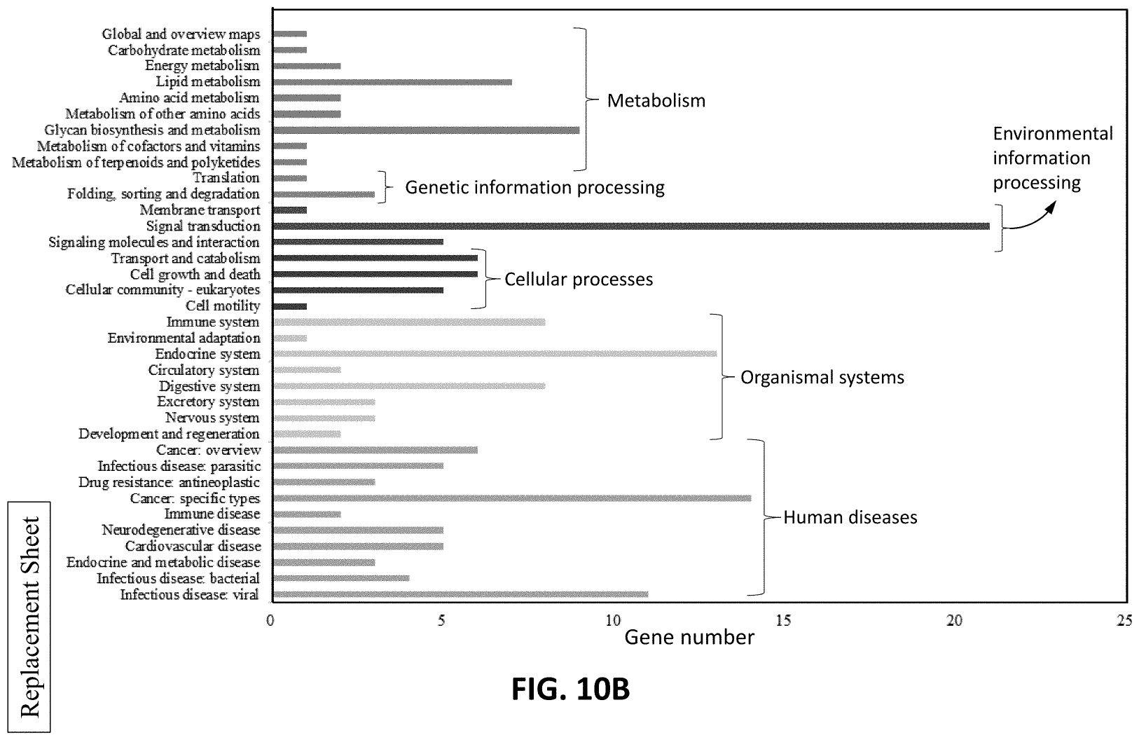

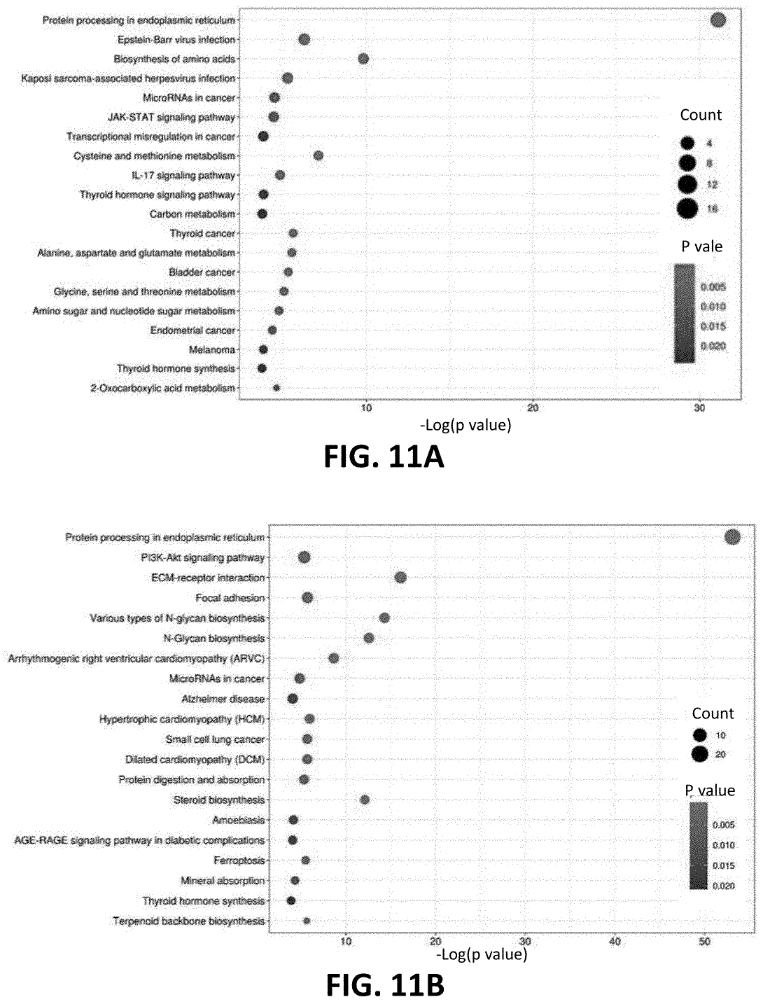

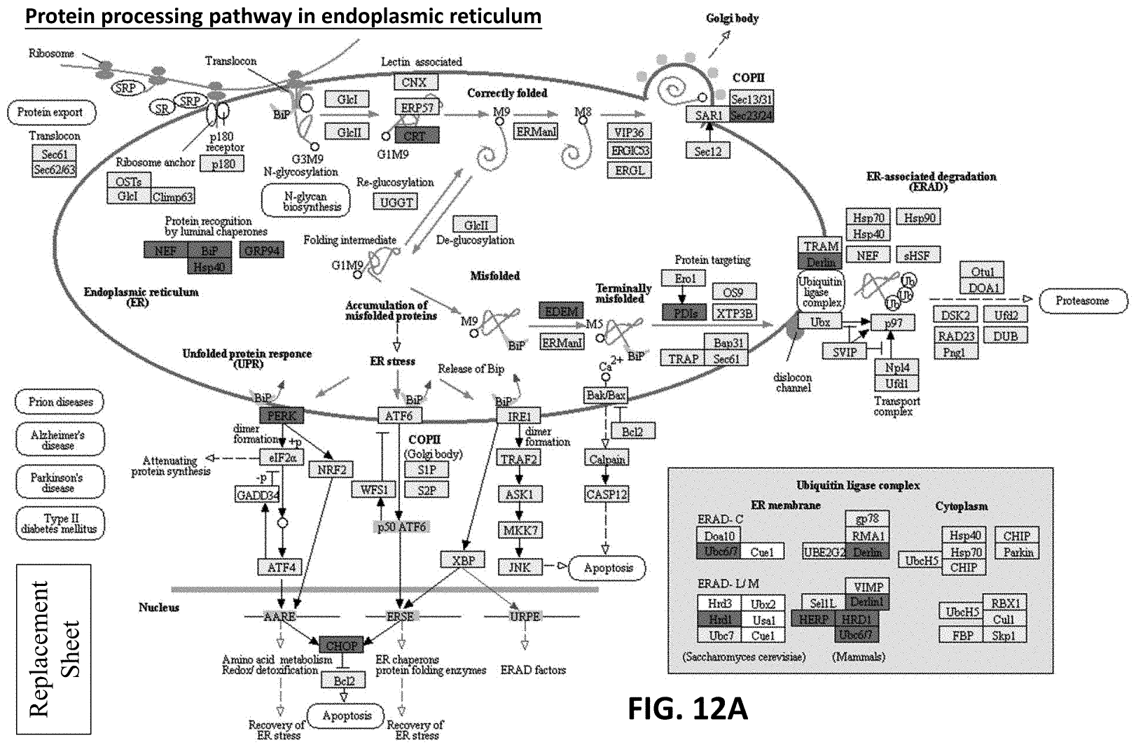

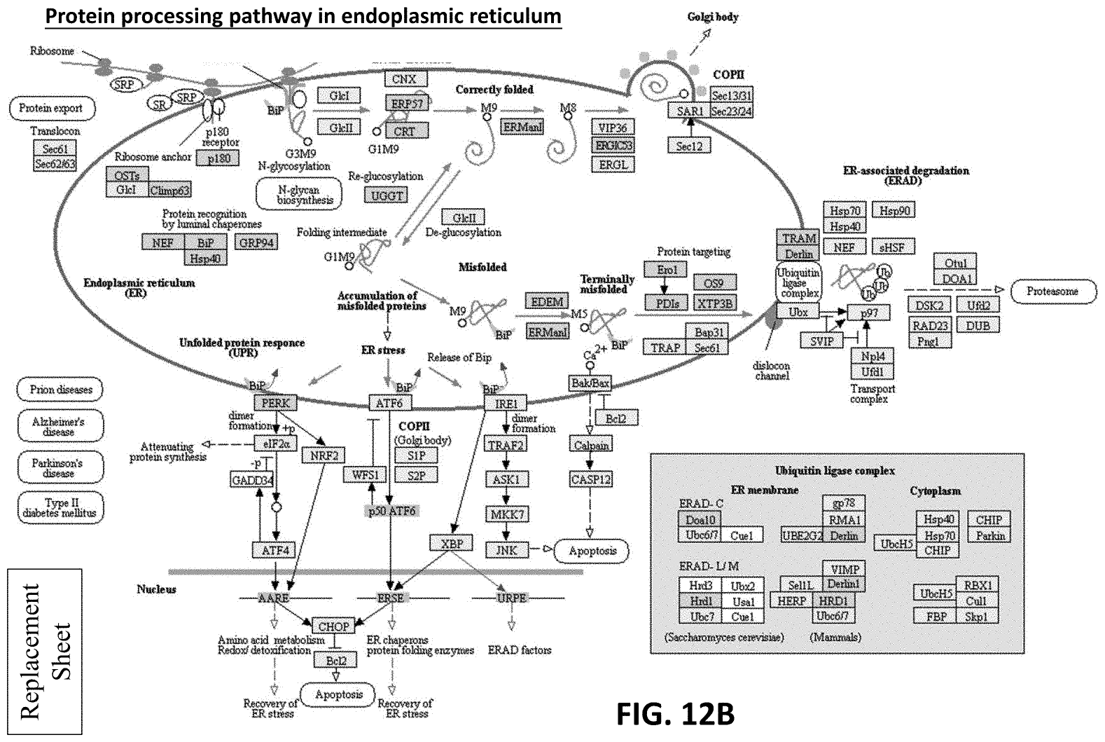

The KEGG pathway enrichment analysis was also performed to classify the foregoing 2 differentially expressed gene groups. In the KEGG pathway enrichment analysis, all of the foregoing differentially expressed genes were classified according to different KEGG pathways including “metabolism”, “genetic information processing”, “environmental information processing”, “cellular processes”, “organismal systems”, and “human diseases.” FIGS. 10A to 10B show that the 2 differentially expressed gene groups are most relevant to the signal transduction. What's more, FIGS. 11A to 11B show that the differentially expressed gene group identified from African green monkey kidney Vero cells infected with dengue relative to reference African green monkey kidney Vero cells and the differentially expressed gene group identified from African green monkey kidney Vero cells infected with dengue in combination with chlorophyllide treatment relative to African green monkey kidney Vero cells infected with dengue are both classified into protein processing in endoplasmic reticulum. According to the classification result, FIGS. 12A to 12B show the relevant differentially expressed genes are marked in the protein processing pathway in endoplasmic reticulum, wherein red labels denote positive regulated genes, and green labels denote negative regulated genes.

While the invention has been described in connection with what is considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A method for treating or preventing virus infection, comprising:
administering a therapeutically effective concentration of chlorophyllide to a subject in need thereof.

2. The method as claimed in claim 1, wherein the virus is flavivirus.

3. The method as claimed in claim 1, wherein the virus is alphavirus.

4. The method as claimed in claim 1, wherein the virus is yellow fever virus, Japanese encephalitis virus, dengue virus, West Nile virus, Zika virus, chikungunya virus, or severe acute respiratory syndrome coronavirus 2.

5. The method as claimed in claim 1, wherein the virus is dengue virus, Zika virus, or chikungunya virus.

6. The method as claimed in claim 1, wherein the virus is Zika virus, or chikungunya virus.

7. The method as claimed in claim 6, wherein the therapeutically effective concentration is 25 μg/mL to 100 μg/mL.

8. A method for treating or preventing virus infection, comprising:
administering a product obtained by treating a plant leaf extract with chlorophyllase to a subject in need thereof, wherein the product comprises a therapeutically effective concentration of chlorophyllide.

9. The method as claimed in claim 8, wherein the product is produced by a method comprising:
providing plant leaves;
performing extraction on the plant leaves with a solvent to obtain a crude extract; and
treating the crude extract with chlorophyllase to obtain the product.

10. The method as claimed in claim 9, wherein the solvent is ethanol or hexane.

11. The method as claimed in claim 8, wherein the virus is flavivirus.

12. The method as claimed in claim 8, wherein the virus is alphavirus.

13. The method as claimed in claim 8, wherein the virus is yellow fever virus, Japanese encephalitis virus, dengue virus, West Nile virus, Zika virus, chikungunya virus, or severe acute respiratory syndrome coronavirus 2.

14. The method as claimed in claim 8, wherein the virus is dengue virus, Zika virus, or chikungunya virus.

15. The method as claimed in claim 8, wherein the virus is Zika virus, or chikungunya virus.

16. The method as claimed in claim 15, wherein the therapeutically effective concentration is 25 μg/mL to 100 μg/mL.
